(19) United States
(12) Patent Application Publication
(10) Pub. No.: US 2024/0423503 A1
(43) Pub. Date: Dec. 26, 2024

(54) Human Gait parameter and Health Information Extraction using Floor-Mounted Geophone Sensors

(71) Applicant: The Board of Trustees of the Leland Stanford Junior University, Stanford, CA (US)

(72) Inventors: Yiwen Dong, Stanford, CA (US); Hae Young Noh, Atherton, CA (US)

(21) Appl. No.: 18/745,010

(22) Filed: Jun. 17, 2024

Related U.S. Application Data
(60) Provisional application No. 63/522,384, filed on Jun. 21, 2023.

(51) Int. Cl. A61B 5/11; G16H 50/30

(52) U.S. Cl. CPC A61B 5/112; A61B 5/1126; G16H 50/30; A61B 2562/0204

(57) ABSTRACT

A gait analysis is provided by passively sensing footstep-induced floor vibrations during walking using vibration sensors mounted on, within or under a floor. The approach is non-intrusive, scalable, and perceived as more privacy-friendly, making it suitable for continuous gait health monitoring in daily life. The floor vibration-based gait analysis framework estimates various gait parameters, including temporal parameters (step, stride, stance, swing time) and spatial parameters (step length, width, angle), and extracts gait health indicators (cadence/walking speed, left-right symmetry, gait balance, initial contact type).

### CROSS-REFERENCE TO RELATED APPLICATIONS

This application claims priority from U.S. Provisional Patent Application 63/522,384 filed Jun. 21, 2023, which is incorporated herein by reference.

### STATEMENT OF GOVERNMENT SPONSORED SUPPORT

This invention was made with Government support under contract 2026699 awarded by the National Science Foundation. The Government has certain rights in the invention.

### FIELD OF THE INVENTION

This invention relates to gait information extraction using floor sensors.

### BACKGROUND OF THE INVENTION

Gait analysis is a key component in the diagnosis, progressive tracking, and rehabilitation of musculoskeletal or neuromuscular disorders. It typically involves estimating spatiotemporal gait parameters and extracting health-related indicators, such as step time, length, symmetry, and balance. For example, existing studies have shown that estimating spatiotemporal parameters is critical for diseases such as dementia, cerebral palsy, and muscular dystrophy, which leads to treatments that can delay the progression and extend patients' life. In addition, gait parameters are also essential for tracking the progress of physical rehabilitation from injuries and stroke, which enables timely interventions that accelerate the process of recovery. Moreover, balance and symmetry have been shown to be critical for fall prediction for the elderly. According to the Center of Disease Control (CDC), every one in four older adults falls in the U.S., and millions of them are hospitalized as a result of falls. Quantitative measurements of gait health can help individuals understand their health status and safety risks, leading to improved life quality.

Traditional gait analyses are typically conducted in gait clinics, approaches include direct observation by medical staff, force plates, electromyography, and infrared cameras for motion capturing. These existing approaches can achieve high accuracy in well-calibrated environments but are unsuitable for continuous monitoring in daily life. This is because they require professionally trained staff to operate and only provide sporadic measurements at the time of in-person visits. To overcome this limitation, other studies have developed portable cameras, wearable devices, pressure mats, and radio frequency (RF)-based systems that enable more frequent and continuous gait health monitoring in non-clinical settings. However, they have raised privacy concerns and operational limitations such as direct line-of-sight, having to carry/charge devices, and dense sensor deployment, which prevents them from ubiquitous usage in daily life. The present invention addresses these concerns and provides new technology in the diagnosis, progressive tracking, and rehabilitation of musculoskeletal or neuromuscular disorders.

### SUMMARY OF THE INVENTION

In one embodiment, the invention is characterized as a gait analysis method using footstep-induced floor vibrations is provided. The method distinguishes the steps of capturing floor vibration signals using two or more vibration sensors distributed and mounted on, within or under a floor. The floor vibrations are footstep-induced floor vibrations caused by a person walking across the floor. It would be possible to executed the method with only one vibration sensor. Temporal gait parameters are predicted using a computer-implemented floor temporal estimation model with the captured floor vibration signals as input to the floor temporal estimation model. Examples of temporal gait parameters are step length, stride length, stance time, swing time, single stance time, double-support time, or a combination thereof. Spatial gait parameters are predicted using a computer-implemented spatial parameters estimation model with the captured floor vibration signals as input to the floor spatial parameters estimation model. Examples of spatial gait parameters are step length, stride length, step width, step angle, or a combination thereof. Using both the predicted temporal gait parameters and the predicted spatial gait parameters as input to a computer-implemented gait health indicator extraction model, gait health indicators are then predicted for the person. Examples of gait health indicators are cadence, speed, symmetry, balance, initial contact, or a combination thereof. The gait health indicators are used to generate a personalized gait profile for the person to understand a gait health compared with an average gait from a group of people.

In another embodiment the invention is characterized as a method using floor-mounted vibration sensors (geophones) to capture the floor vibrations generated by footsteps during walking. The collected data is then processed using machine learning algorithms to estimate various gait parameters, including temporal parameters (step, stride, stance, swing time), and spatial parameters (step length, width, angle). In addition, our method extracts additional health indicators (cadence, left-right symmetry, gait balance, initial contact type), which are important for gait abnormality detection and characterization.

Embodiments can be applied in gait monitoring for both clinical and non-clinical settings. In clinics, it can be used for rapid and quantitative functional/mobility/balance scoring, abnormal gait detection, surgical planning, the planning of other therapeutic interventions for neuromuscular/musculoskeletal diseases, and better design of orthosis. In non-clinical settings, our system is a smart home assistant that can be used at home/eldercare facilities for fall risk assessment and detection, progressive tracking of rehabilitation, early discovery of neuromuscular/neurological diseases through gait, the improvement of sports performances, and daily activity level tracking.

The advantages of the approach include that it is non-intrusive, scalable, and perceived as privacy-friendly. First, no direct contact is needed between the user and the sensor because the sensors are attached to the floor and furniture and operate passively. The approach is scalable because the sensors can be sparsely deployed (up to 20 m sensing range). In addition, vibration naturally encodes people's private information through structural mediums, making users less concerned about privacy and minimizing interference with their daily activities.

Therefore, the approach provides continuous monitoring of an individual's gait parameters and health-related information at home, allowing for early detection of health issues, evaluation of the effectiveness of rehabilitation programs, and provide timely interventions when needed.

### BRIEF DESCRIPTION OF THE DRAWINGS

Drawings are presented in black/white or gray-scale. Reader is referred to Appendix A in the priority document for color-interpretation of the drawings.

FIGS. 1A-B show according to an exemplary embodiment of the invention the use of vibration sensors to capture the floor vibrations generated by footsteps during walking (FIG. 1A). Algorithms have been developed to analyze these vibrations, which produces estimates on spatial, temporal gait parameters and gait health indicators (represented by the three different colored (now gray scale) lines in FIG. 1B). The outcome of this approach is a personalized gait profile for individuals to understand their gait health compared with the average gait from all people (gray dashed line).

FIG. 2 shows according to an exemplary embodiment of the invention an illustration of temporal gait parameters with respect to footstep-induced structural vibration signals on a wooden floor: the foot strike and foot off divide the gait cycle into stance and swing phases. A normal foot strike occurs at the valley right before the high-frequency peak, while a normal foot-off occurs at the peaks around the natural frequency range of the structure.

FIG. 3 shows according to an exemplary embodiment of the invention an overview of the spatial gait parameters estimated in embodiments of this invention.

FIG. 4 shows according to an exemplary embodiment of the invention heterogeneity in wave propagation velocity observed based on (left) wave propagation distance and (right) wave propagation time. Distance and time have different trends, meaning that the velocities vary among different footstep locations.

FIG. 5 shows according to an exemplary embodiment of the invention representative examples on the wavelet domain of floor vibration signals under three types of initial contacts.

FIGS. 6A-B show according to an exemplary embodiment of the invention floor vibrations induced by a person walking on two types of floors (FIG. 6A) concrete, (FIG. 6B) wooden floor. The major gait events are captured in different dominant frequency ranges by the wavelet coefficient map.

FIG. 7 shows according to an exemplary embodiment of the invention visualization of velocity profile models for wooden and concrete floors, respectively. Overall, the velocity profile correlates well with the structural layout—the symmetrical wooden floor structure has a symmetrical velocity profile, and the asymmetrical concrete spans are reflected in the asymmetrical velocity profile. The velocity in concrete is generally higher than that in wood.

FIG. 8 shows according to an exemplary embodiment of the invention the framework of gait analysis through footstep-induced floor vibrations has four modules (red boxes), including: 1) sensing and pre-processing, 2) floor-agnostic temporal parameter estimation, 3) floor-agnostic spatial parameter estimation, and 4) gait health indicator extraction. The data flow pipeline is represented by back solid lines. The outcomes of the framework are highlighted as green-colored (not gray-scale) text.

FIG. 9 shows according to an exemplary embodiment of the invention floor-mounted geophone sensors for footstep-induced floor vibration sensing (left). A sample series of detected footsteps through peak-picking of the wavelet coefficients (right).

FIGS. 10-11 show according to an exemplary embodiment of the invention an overview of the temporal gait parameter estimation process.

FIG. 12 shows according to an exemplary embodiment of the invention a sample walking trajectory estimated from the heel strike locations.

FIGS. 13A-D show according to an exemplary embodiment of the invention an experimental setup for (FIG. 13A) vibration sensors mounted at the edge of the walkway, (FIG. 13B) Vicon motion capture system with lower body locomotion for ground truth collection, (FIG. 13C) wooden floor test layout, and (FIG. 13D) concrete floor test layout.

FIG. 14 shows according to an exemplary embodiment of the invention gait health indicator estimation error rate (RMSPE) for 20 subjects.

FIG. 15 shows according to an exemplary embodiment of the invention 4 typical gait profiles from 20 subjects. The axes in each profile represent the estimated temporal gait parameters (green, now gray-scale), spatial gait parameters (red, now gray-scale), and gait health indicators (blue, now gray-scale). The gray dotted line represents the mean value among all subjects. The indicated temporal, spatial and health circled-part lines are at the same position in all four profiles.

FIG. 16 shows according to an exemplary embodiment of the invention both spatial and temporal parameter estimation results are consistent across two types of floors. Embodiments of the system have a significant improvement over the baseline in which there is no floor adaptation when migrating from wooden to concrete floor.

### DETAILED DESCRIPTION

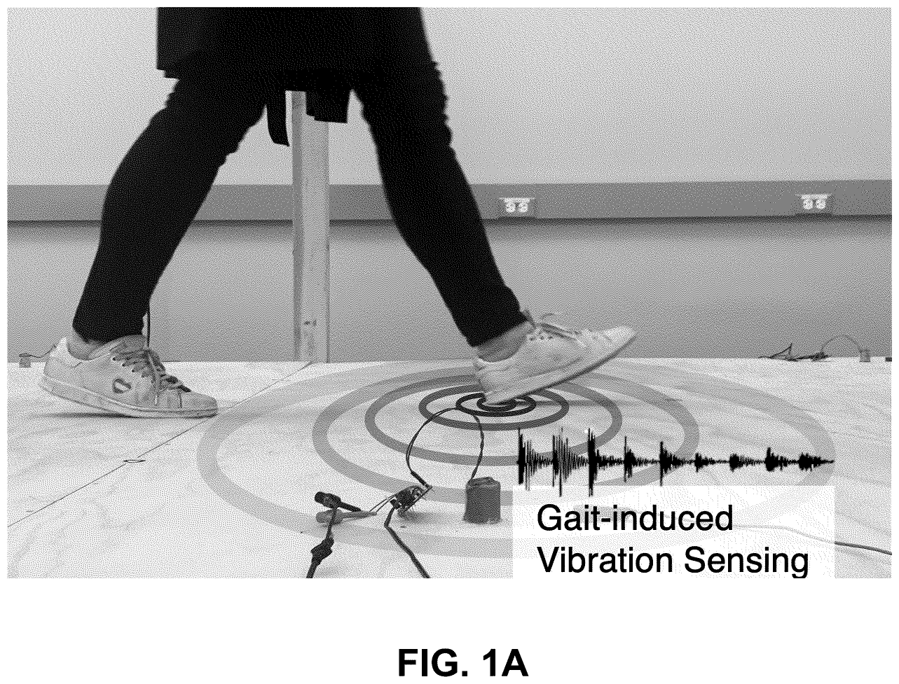

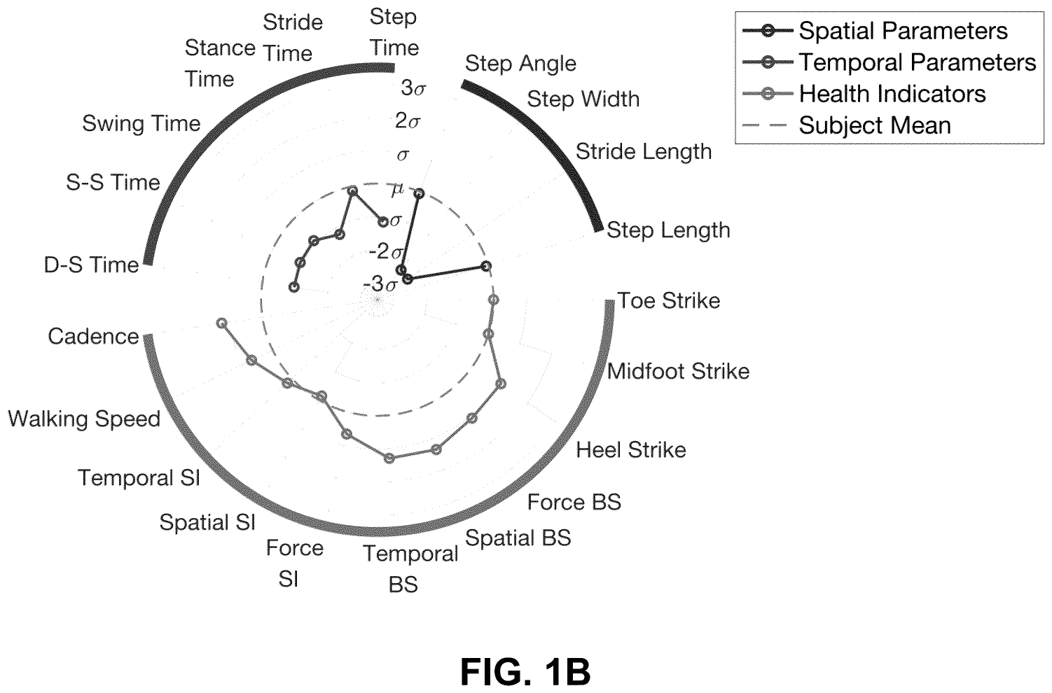

A novel sensing approach for gait analysis is provided using footstep-induced floor vibrations to enable non-intrusive continuous monitoring of gait health. The method uses vibration sensors (e.g., geophone and/or accelerometer) to capture the floor vibrations generated by footsteps during walking. The collected data is then processed using machine learning algorithms to estimate various gait parameters, including temporal parameters (e.g., step, stride, stance, swing time) and spatial parameters (e.g., step length, width, angle). In addition, the method extracts additional gait health indicators (cadence, left-right symmetry, gait balance, initial contact type), which are important for gait abnormality detection and characterization. The main idea of this approach is that individuals' gait induced floor vibrations, being captured by the vibration sensors placed on the floor surface (see FIG. 1A). By analyzing these vibration signals, one could then infer a person's gait profile in terms of spatio-temporal gait parameters and gait health indicators (FIG. 1B).

The advantages of the sensing approach include that it is non-intrusive, scalable, and perceived as privacy-friendly. First, no direct contact is needed between the user and the sensor because the sensors are attached to the floor and furniture and operate passively. The approach is scalable because the sensors can be sparsely deployed (up to 20 m sensing range). In addition, vibration naturally encodes people's private information through structural mediums, making users less concerned about privacy and minimizing interference with their daily activities. Therefore, the approach provides continuous monitoring of an individual's gait parameters and health-related information at home. This allows for early detection of health issues, evaluation of the effectiveness of rehabilitation programs, and timely interventions when needed.

A main challenge in developing this approach is the effect of different floor structures on the vibration signals. For example, the surface roughness, material properties, beam/column dimensions, and layouts are different across various buildings, making it difficult to develop an algorithm that is generalizable to different types of floors. To overcome the challenge, the inventors characterized the structural vibrations for various floor types and extract features that are insensitive to the floor but sensitive to gait parameters. To this end, this approach can be easily adapted to a variety of buildings, including homes, hospitals, and eldercare facilities.

The core contributions of this invention are:
The development of a gait parameter estimation and health information extraction framework using footstep-induced floor vibrations, which is the most in-depth and comprehensive vibration-based gait analysis to date.
The characterization of the footstep-induced floor vibration across various types of floor structures to design an algorithm that is generalizable to a variety of types of buildings with different floor structures.
An evaluation of this approach through a real-world experiment with 20 subjects across two types of floor structures and achieve promising accuracy in estimating gait parameters and extracting health information.

To evaluate the approach, field walking experiments were conducted with 20 subjects from various age groups across two types of floors, with both vibration sensing and Vicon 96 Motion Capture systems (for ground truth only). Through the experiment, 12,231 gait cycles were captured to estimate gait parameters and gait health indicators. This approach has achieved an average of 91.5% (RMSE 0.08 s), 85.6% (RMSE 0.38 m), 92.3% (RMSPE 7.7%) 99 accuracy in estimating temporal, spatial parameters, and health information, respectively.

In the rest of the description, the floor vibration data is first characterized among various floor types to understand its relationship with the gait parameters and gait health indicators. Then, the gait analysis framework is presented by describing the details of the algorithm to estimate gait parameters and extract gait health indicators. Next, the framework is evaluated through a real-world experiment. The results demonstrate the effectiveness of the approach in accurately estimating gait parameters and extracting gait health information.

### Characterization of Footstep-Induced Floor Vibrations for Gait Analysis

In this section, the floor vibrations induced by gait are characterized to understand their relation to gait characteristics. First, the physical insight of footstep-induced floor vibration for gait analysis is introduced. Then, the relationship between spatio-temporal parameters and floor vibration was established. Finally, the vibration signals from different floor structures were characterized to understand its effect on gait analysis.

### Physical Insight Behind Footstep-Induced Floor Vibrations

The main physical insight that behind floor vibration-based gait analysis is as follows: when a person is walking around, each footstep exerts a short-duration force onto the floor, which causes a small deflection on the floor surface. This deflection changes the internal stress of the floor slabs—both the shear force and the bending moment around the impact location increases. The change of force condition breaks the force equilibrium and results in dynamic responses of the floor to restore its equilibrium. These footstep-induced repeated deflection-restoration cycle is described as “vibrations” of the floor structure. As the ensuing vibration waves propagate through the floor, they can be measured by vibration sensors mounted at the floor surface at a distance away from the footstep locations. The vibration sensors transform the vertical displacements of the floor into electrical voltage time series. Since the variation in the footstep forces leads to different floor responses, the collected vibration signals were analyzed to infer human gait characteristics.

### Relationship Between Gait Characteristics and Footstep-Induced Floor Vibrations

Gait parameters are quantitative measurements of human walking in terms of duration, location, and characteristics, which are components in gait analysis. In this subsection, the relationship between temporal and spatial gait parameters and floor 132 vibration signals is characterized.

### Temporal Gait Cycle Characteristics

### Background of Gait Cycle and Temporal Parameters

A gait cycle is defined as the duration between a foot strike and the subsequent foot strike of the same foot. A typical gait cycle focuses only on one leg and has two primary phases: the stance phase and the swing phase. The stance phase is the duration when the foot is in contact with the floor; The swing phase is the duration when the foot is swinging in the air. The stance and swing phases can be separated by two major gait events: 1) a foot strike, which is the onset of the stance phase, and 2) a foot off, which is the onset of the swing phase. When considering the opposite foot, one gait cycle can be further divided as the alternation of double support and single support of two feet. The double support is when both feet are on the floor and the single support is when one foot is swinging in the air.

The temporal gait parameters are defined based on the duration of stance/swing phases and double/single support time. Estimating the duration of these phases helps to identify potential gait abnormalities. For example, a shorter stance time on one leg indicates asymmetrical gait and difficulty in maintaining balance while walking, which may lead to an increased risk of falls.

### Relationship Between Temporal Parameters and Floor Vibrations

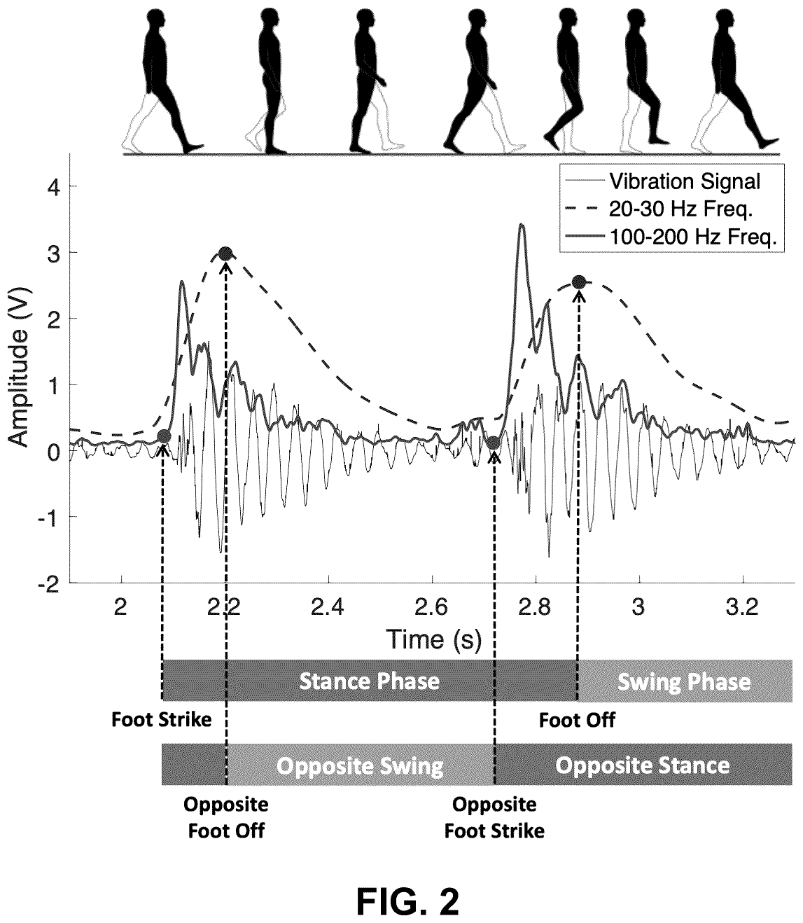

To estimate temporal gait parameters from floor vibration signals, the footstep-induced floor vibration is characterized with respect to the time when critical gait events happen. FIG. 2 illustrates how different frequency ranges in the floor vibration signals correspond to the gait events and phases.

As observed in FIG. 2, the floor vibration induced by a gait cycle has two consecutive footstep impulses from the left and right foot. This is because the gait cycles from the left and right foot overlap while walking. After the first footstep impulse, the second impulse happens when the person alters the support foot to another, which is marked as the opposite foot strike. In addition, the inventors observed that the gait events occur at special points in their dominant frequency ranges. Specifically, the foot strike excites a higher frequency in vibration signals than the foot off because of the impulsive footstep force at the point of contact. The foot-off occurs at the peaks of the low-frequency range around the natural frequency of the floor. This aligns with the inventors' intuition that the foot-off signifies the beginning of free vibration (i.e., floor vibrations when the foot swings in the air), where the natural frequency component reaches the maximum and starts to attenuate. This allows us to segment the gait cycle into phases and extract temporal gait parameters, which will be discussed further infra.

### Spatial Gait Cycle Characteristics

### Background of Spatial Parameters

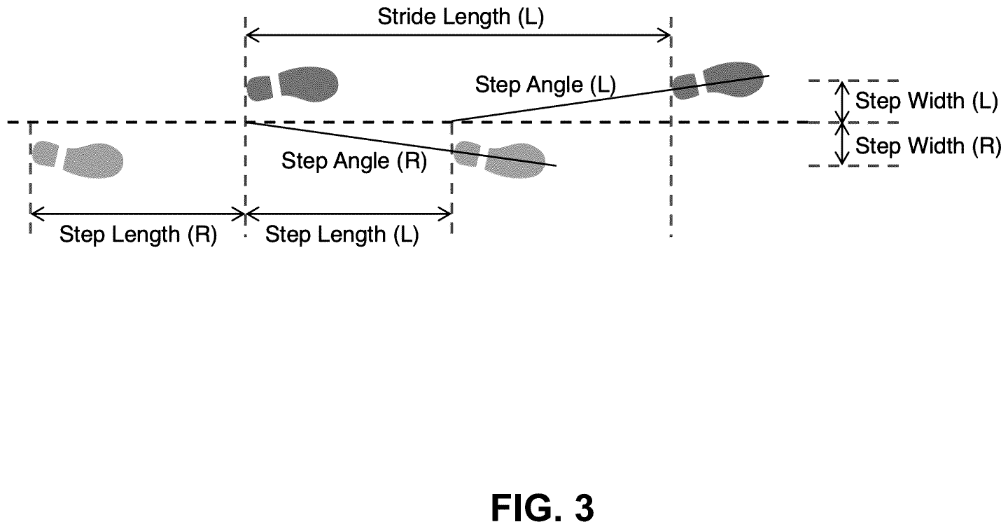

The spatial parameters are defined based on the location of the footsteps. As described in FIG. 3, a person takes four consecutive footsteps when walking in a straight line. The dashed center line between the left and right footsteps represents the walking trajectory. In this case, the step length is defined as the distance between left and right foot strikes perpendicular to the walking trajectory. Similarly, the stride length is defined as the distance between the two adjacent strikes from the same foot, perpendicular to the walking trajectory. On the other hand, the step width is defined as the distance between the center of the footstep and the projected footstep center to the walking trajectory. The step angle is the angle of this projection by setting the previous foot strike location as the origin.

In this invention, the spatial parameters were estimated based on the footstep location because all the spatial parameters mentioned above can be calculated based on the location.

### Characterizing the Floor Heterogeneity for Spatial Parameter Estimation

To localize the footsteps, previous studies have explored the time-difference-of-arrival (TDoA) method, which provides the lowest error so far for footstep localization on floor structures around 0.5 meters. However, this error is too large for spatial gait parameter estimation because the average step length of an adult is around 0.5 meters, meaning that the existing work has a 100% error rate. In fact, the main research barrier that prevents accurate vibration-based footstep localization is heterogeneous wave propagation velocity in the floor structures, leading to high uncertainty in estimated locations. This has long been a challenging problem to address because the wave propagation velocity is typically unknown and is affected by complex factors, including material properties, defects/cracks in the structure, and the properties of the connections between structural components. As a result, when a person's footstep location changes, the underlying structural property also changes, resulting in a different wave propagation velocity.

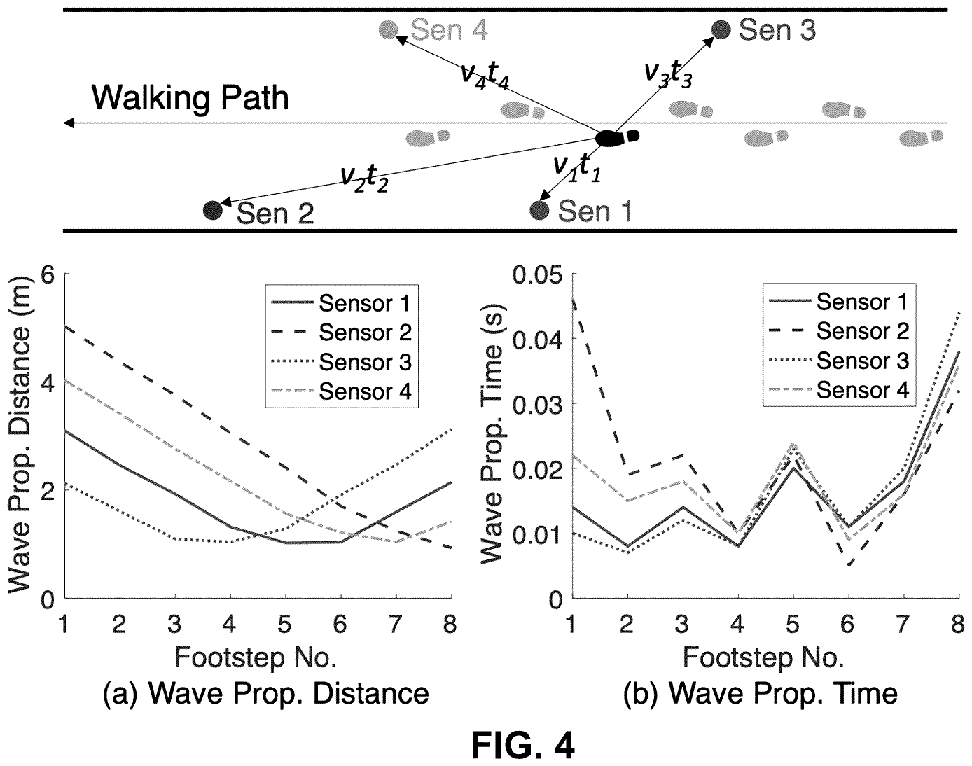

Therefore, in this invention, the inventors characterized the wave propagation velocity by dividing the wave propagation distance by the propagation time, to calibrate the structure for higher accuracy in spatial parameter estimation. Specifically, when a person walks across the walkway, temporarily-installed cameras can be used to measure the time and distance between the footstep location and the location of each sensor, as shown in FIG. 4 (left). With the distance and time between the footstep and sensor i, one can estimate the wave propagation velocity by:

$${v}_{i} = \frac{{d}_{i}}{{t}_{i}} \qquad ( 1 )$$

FIG. 4 (right) shows the changes in wave propagation distance and time as the person walks by. Since the sensors are mounted at different locations, the wave propagation distance reaches the minimum from left to right as the person is the closest to each sensor (FIG. 4, left). However, the wave propagation time follows a different trend, where the minimum does not align with the minimum distance. This means that the velocity varies across different locations on the floor. By taking the division between distance and time, the inventors found that the velocity is between 30 m/s and 300 m/s on the testing walkway, which can be the main source of uncertainty in footstep localization.

In addition, the wave propagation direction also affects the velocity. For example, for the 5th footsteps in FIG. 4 (highlighted as black in the upper diagram), the wave traveling velocity has a range of ˜70-130 m/s among these four sensors. Compared to the velocity range across different locations (30-300 m/s), the effect of different wave propagation directions is less significant. Therefore, it was assumed that the velocity at each footstep location is the mean of all sensors during the calibration because it is not practical to estimate the velocity in a space with an unlimited number of locations and directions. With this assumption, one can then calibrate the floor using a spatial velocity profile, which reduces the uncertainty and leads to a more accurate footstep localization.

### Gait Health Characteristics

### Background of Gait Health Indicators

The gait health indicators are quantitative measurements herein defined based on estimations of spatio-temporal parameters and forces, combined with the initial contact types. In the following, the background of gait health indicators being estimated in this invention is summarized, including 1) cadence/walking speed, 2) symmetry, 3) balance, and 4) initial contact type.

Cadence is the number of steps taken per minute while walking, which is 60 times the step frequency (steps per second). Walking speed is the amount of distance traveled at a given time, typically represented as (meter per second or kilometer per hour). A slower walking speed is typically associated with an increased functional decline in walking. Increasing cadence has been shown to improve balance and levels of physical activity.

Symmetry refers to the similarity and coordination between the left and right sides of the body while walking. A lack of symmetry can cause an uneven distribution of weight which can increase fall risks and lead to musculoskeletal pain or injury. Existing studies have defined symmetry mainly through symmetry ratio and index. Both compare the difference between left and right foot. The symmetry ratio is calculated as the quantity of the left foot divided by the right foot. The symmetry index (SI) is computed as the percentage difference between the left and right foot measurements. The symmetry estimation approach of this invention is based on spatial, temporal parameters, and force-related estimates, which will be introduced infra.

While gait symmetry is about the equality of left and right foot movements during walking, gait balance refers to maintaining stability and control during walking, where a person adjusts postures and movements in response to changes in the walking conditions or external disturbances. Good balance is necessary to preserve stability and prevent falls. Gait balance is typically assessed through various tests in clinical settings, including the Berg 242 Balance Scale (BBS), Mini-BESTest, and backward walking, which involves evaluation of the quality of completion in several physical tasks. To assess gait balance during realistic walking scenarios, previous studies typically used the variability in step width and time, which correlate well with the balance measurements in the clinical tests. Therefore, the inventors herein defined a balance score based on the variability of the footsteps, which will be introduced infra.

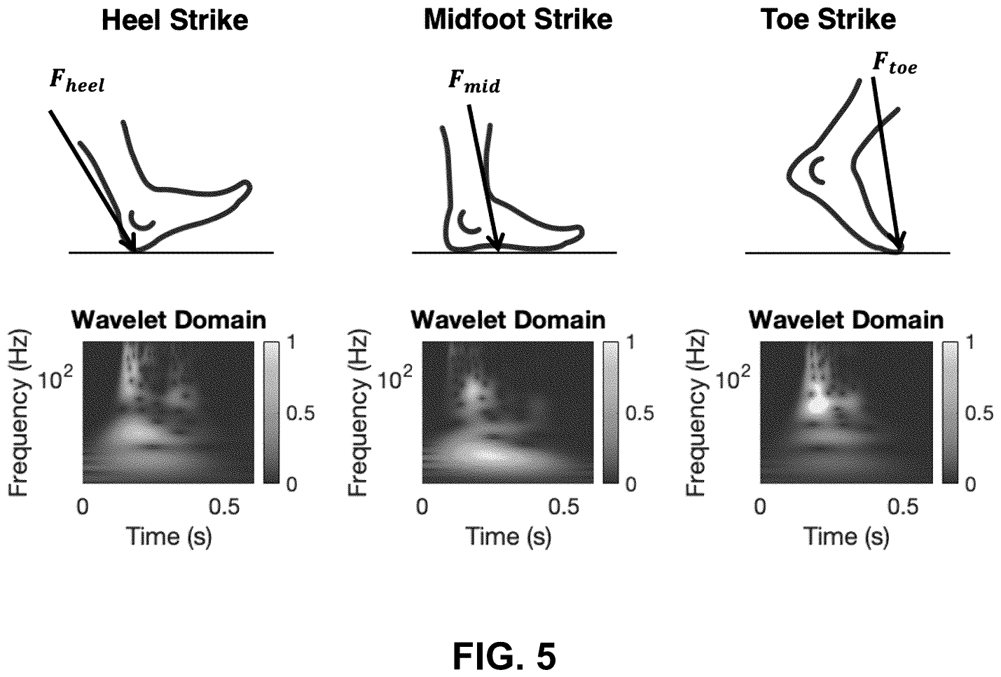

Initial contact type refers to the pattern when the foot contacts the floor during the foot strike. There are three main types of initial contact: heel strike, toe strike, and midfoot strike (see FIG. 5). The type of initial contact is determined by muscle activation and affects the force transmission through the body during walking. For example, a toe strike may be caused by excessive gastrocnemius contraction and can lead to a greater load on the Achilles tendon, and a heel strike typically results in a larger moment on the knee joint. Understanding the individual's initial contact type is important to assess the functional ability in walking.

### Characterizing the Floor Vibration for Initial Contact Types

Different types of initial contact lead to distinct patterns in floor vibrations, which are shown in the wavelet domain 258 plots in FIG. 5. The heel strike induces a higher frequency at the initial contact and a lower frequency during the later progression of the foot. The midfoot strike leads to a lower frequency than that of the heel strike because the footstep force is less impulsive. In contrast, the toe strike results in mainly high-frequency components due to the lack of foot progression on the floor.

### Effect of Floor Types on Floor Vibration-Based Gait Analysis

To address the core research challenge of floor type variations, the inventors characterized the vibration signals from various types of floor structures to understand its effect on gait analysis. Specifically, the inventors analyzed and discussed the floor type influence on temporal and spatial parameter estimation, respectively.

### Floor Type Influence on Temporal Parameter Estimation

### Time-Frequency Analysis of the Floor Influence

The inventors formulated the influence of floor types using structural dynamics by assuming the floor is a linear time-invariant system. Through time-frequency analysis of a dynamical system:

$$M \ddot{u} ( t ) + C \dot{u} ( t ) + Ku ( t ) = F ( t ) \qquad ( 2 )$$

where u(t) is the floor displacement, M is the floor mass matrix,

C=diag[2ξⱼωⱼ]

is the damping matrix, K is the stiffness matrix, F(t) is the footstep force. In the frequency domain, one can write:

$$ℱ \{ u ( t ) \} = H ( ω ) ℱ \{ F ( t ) \} ⇒ Y ( ω ) = H ( ω ) X ( ω ) \qquad ( 3 )$$

where Y(ω) is the frequency spectrum of the floor vibration, X(ω) is the Fourier transform of the footstep force, and the influence of the floor structure is encoded in the transfer function H(ω). Based on modal decomposition, each element in the transfer function hⱼ(x, l) of mode j with given sensor location x and footstep location l can be written as:

$${h}_{j} ( x , l ) = {ϕ}_{jx} {FRF}_{j}^{*} ( {ω}_{j} ) {ϕ}_{jl} \qquad ( 4 )$$

In this equation, ϕjx and ϕjl are constants representing mode shapes for a given floor, and the modal frequency response function FRFj*(ωj) has large values only when ωj is close to the modal frequency. Therefore, the dominant frequencies observed in the floor vibration spectrum Y(ω) indicate that footstep force spectrum X(ω) being amplified around the modal frequencies.

Experimental Observation of Temporal Parameters from Floor Vibrations

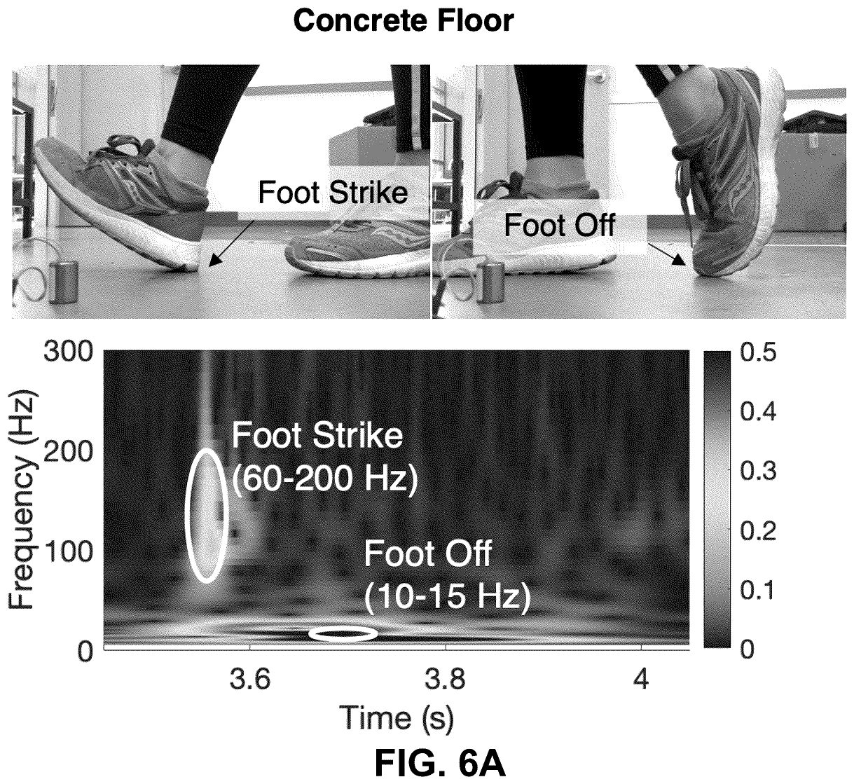

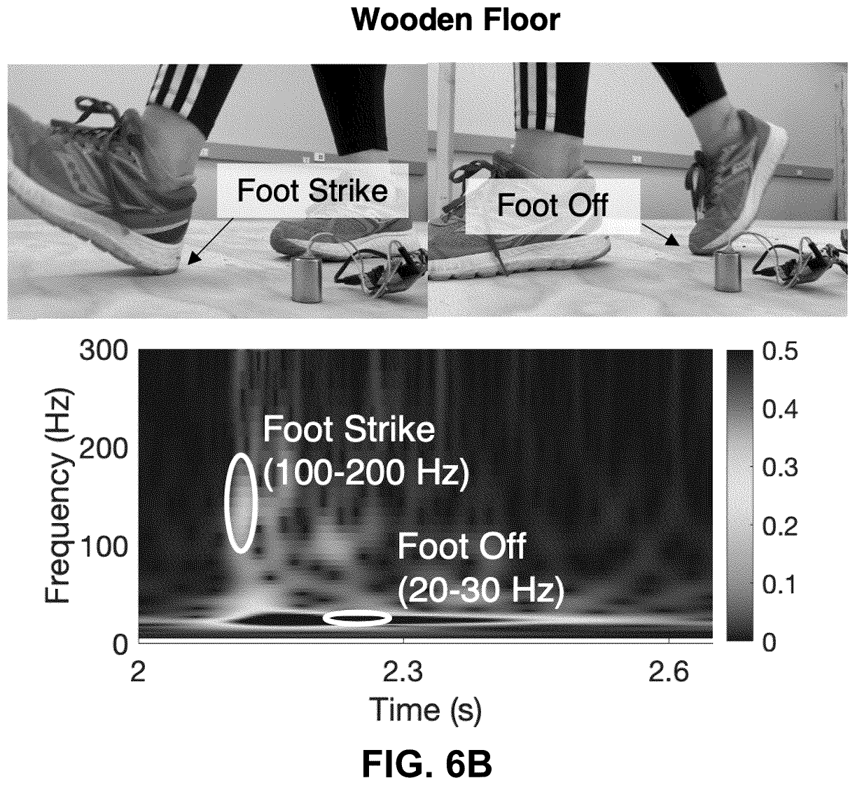

The theoretical analysis was evaluated through a controlled experiment on a wooden and concrete floor. FIG. 6 shows the wavelet coefficient plot of a person's footstep on these floors. While the footsteps for the left and right figures are from the same person wearing the same pair of shoes, the dominant frequencies in the wavelet spectra are significantly different for foot strike and foot off. This observation can be interpreted based on Equation 4: assuming the same person has a consistent footstep force pattern, the spectrum of the footstep force appears to be amplified around the modal frequencies of each structure. Since the modal frequencies of the wooden and concrete floors are different due to their discrepancies in mass, stiffness, and damping ratio, the same footstep force results in different dominant frequencies.

### Floor Type Influence on Spatial Parameter Estimation

### Spatial Floor Heterogeneity Calibration

To overcome the challenge between different floor types, the wave propagation velocity profile was modeled using several initial trials of walking with temporarily installed camera to provide ground truth on footstep location and time. The velocity at each footstep location is computed by distance over time. Specifically, a non-linear regression on the footstep samples was conducted to reduce the effect of outliers and the effect of wave propagation directions. A 4th order polynomial regression model was utilized because of two reasons. First, the cross-section layout of the testing walkways has two spans so at least 4th order polynomial need to be used to model the deformation trend. Secondly, the polynomial order needs to be constrained to achieve a consistent training and validation accuracy that does not over-fit the individual data samples that reflect local defects. The fitted velocity x at location x in our case is described as follows:

$$v ( x ) = {β}_{4} {x}^{4} + {β}_{3} {x}^{3} + {β}_{2} {x}^{2} + {β}_{1} x + {β}_{0} \qquad ( 5 )$$

where βi represents the coefficients estimated during the regression.

### Experimental Observation of the Spatial Velocity Profiles on Two Floors.

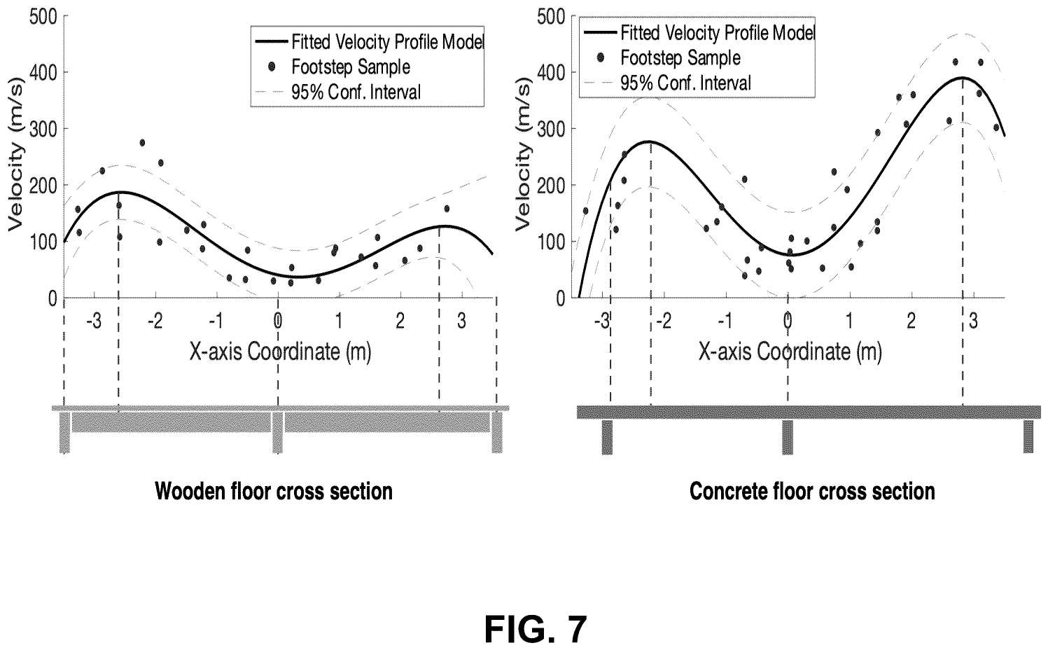

FIG. 7 shows the velocity profile along the longitudinal center line of two types of testing floors. The left is a wooden-framed structure that has two spans of the same lengths. On the right is a concrete floor that has two spans of different lengths. The fitted wave velocity on the concrete floor is significantly higher than on the wooden floor. The estimated velocity profile correlates well with its cross-section layout—the vibration wave travels slower at column locations and faster at the mid-span of the structure.

### Gait Analysis Framework Through Footstep-Induced Floor Vibrations

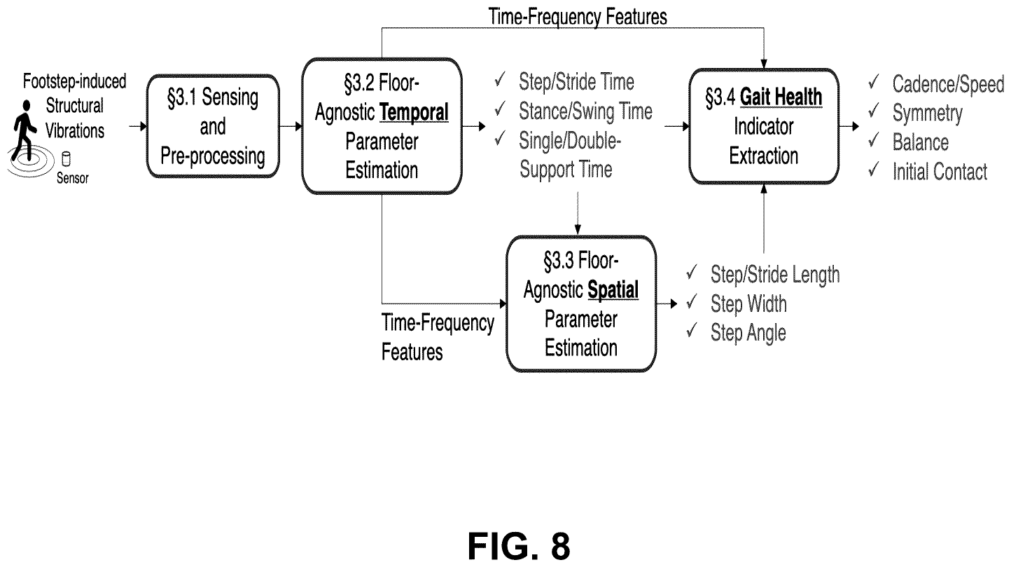

Here the gait analysis framework is introduced, which estimates spatio-temporal gait parameters and extracts gait health indicators using footstep-induced floor vibrations, and is designed to be robust to various floor types (FIG. 8). First, the sensing system is introduced that collects and pre-processes the footstep data. Then, a description follows on how to estimate temporal and spatial gait parameters from various types of floors. Finally, a description follows on how to extract gait health indicators that are commonly used to detect and describe gait abnormalities related to neurological/musculoskeletal disorders.

### Sensing and Pre-Processing

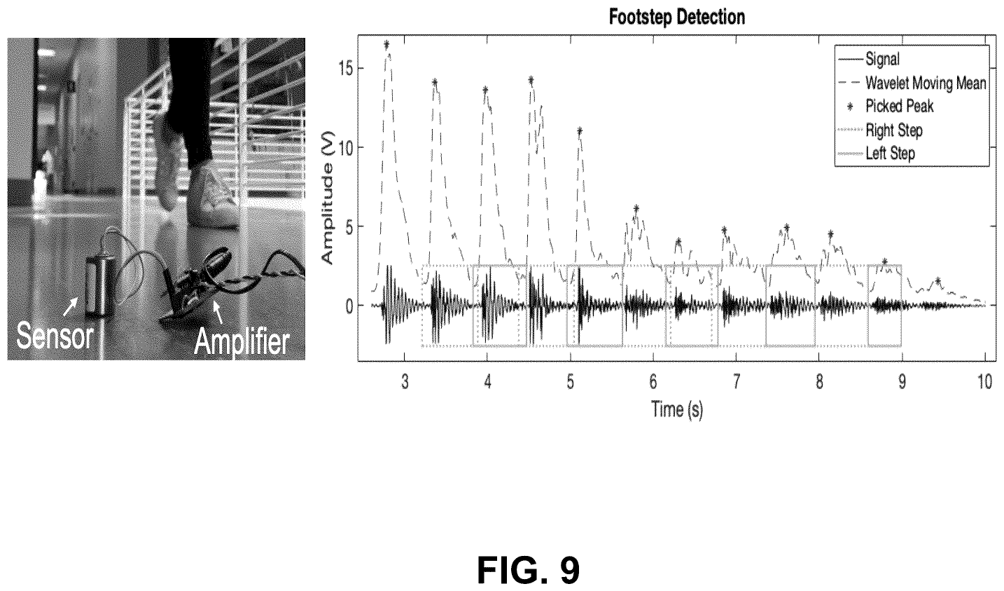

The sensing system uses floor-mounted geophone vibration sensors, as shown in FIG. 9 (left). Geophone sensors are mechanical vibration sensors that convert the velocity of the floor vibrations into an analog voltage signal. The sensors are typically connected to operational amplifiers (op-amp) to increase the signal amplitude and avoid signal clippings. The effective sensing range after amplification is up to 20 meters, which enables sparse sensor deployment at home. For temporal parameter estimation, one sensor is sufficient to produce the results; for spatial parameter estimation, at least three sensors are needed to compare the difference in vibration wave arrival time.

The data pre-processing includes two steps, 1) noise filtering and 2) footstep detection. The former aims to handle electrical and environmental noises, and the latter detects individual footstep-induced impulses from the time series data stream.

The noise filtering process typically involves a lowpass filter and a Wiener filter. The lowpass filter is used to remove high-frequency electrical noises. For temporal parameter estimation and health information extraction, the threshold of the lowpass filter is set to 500 Hz, enabling a Nyquist frequency of 250 Hz, under which the majority of the gait information is preserved. This is determined by comparing between footstep's frequency spectrum and the ambient noise frequency spectrum through preliminary data collection. For spatial gait parameter estimation, the lowpass filter is set to 2500 Hz to compensate for the high wave propagation velocity through the floor medium, enabling an around 10 cm footstep localization resolution through the time-difference-of-arrival (TDoA) method. On the other hand, the Wiener filter is used to reduce environmental noises, which takes in 3-second of signal with only the environmental noise and leverages its frequency spectrum to filter out noise on the signal with combined footstep impulses and environmental noises.

The footstep detection algorithm is developed based on peak-picking of the wavelet coefficients. As shown in FIG. 9 (right), wavelet transform of the entire signal is conducted using the Morlet wavelet, a commonly used wavelet that is efficient in computation and well-suited for time-varying, non-stationary signals. Since footstep-induced vibration signals are impulsive in nature due to the short foot-floor contact duration, the inventors focused on the natural frequency range of typical floor structures (5-30 Hz) in the wavelet coefficients to detect the peaks where these impulses occur. In addition, since footsteps typically occur in groups with repeated patterns in the vibration signals as a person walks by, the inventors set the minimum number of continuous impulses to three so that footsteps are distinguished from other human-induced impulse signals such as item dropping and door opening/closing. When detecting the footsteps, two adjacent footsteps are marked as from the left and right foot, respectively, to prepare for gait symmetry analysis.

### Floor-Agnostic Temporal Parameter Estimation

The temporal gait parameters estimated herein include step time, stride time, stance time, swing time, single-support time, double-support time. These are critical time duration within a gait cycle.

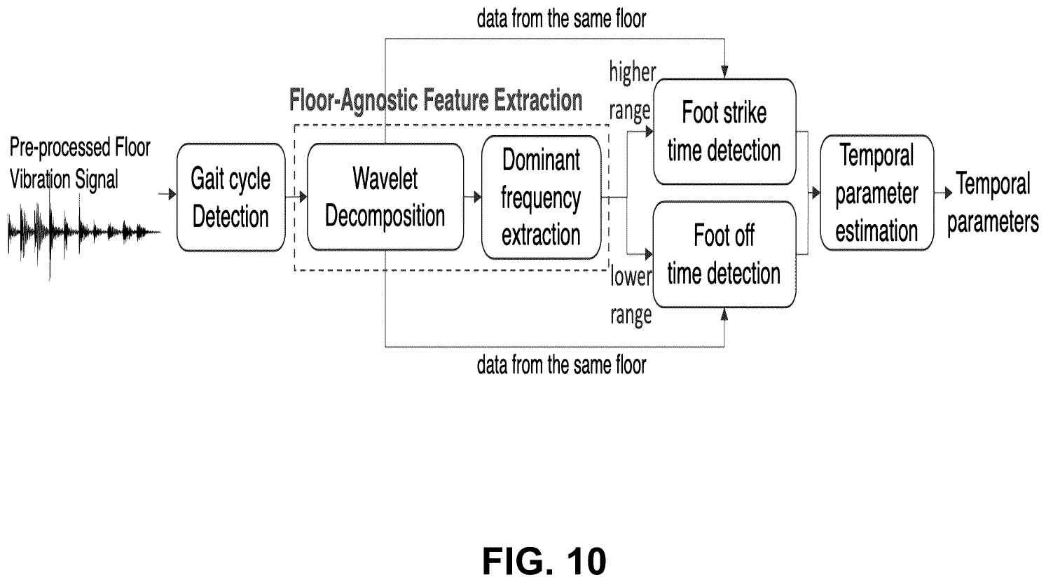

The approach for floor-agnostic temporal parameter estimation has four steps: 1) gait cycle segmentation, 2) floor-agnostic feature extraction, 3) foot strike and off time detection, and 4) temporal parameter estimation. FIG. 10 shows the estimation process.

First, gait cycles are detected by grouping the previously detected individual footsteps. As introduced supra, since a typical gait cycle has two foot strikes (including one foot's strike and the opposite foot's strike), each pair of consecutive left and right footsteps is combined as a gait cycle group.

Then, the floor agnostic features are extracted from the vibration signals, which are the dominant frequency ranges at each gait event. As discussed in FIG. 6, the main difference between the vibration signals from two different floors is the dominant frequency ranges at the foot strike and foot off. The dominant ranges for foot strike is typically around 10-30 Hz, and that of the foot off is around 60-200 Hz, depending on the type of the floor. Therefore, the dominant frequency range is determined by cropping out the first 0-10% of the gait cycle (when foot strike occurs) and 60-70% of the gait cycle (when foot off occurs) to capture the floor difference. When a new trace of footsteps is observed from the same floor, one can then accelerate the process by skipping the dominant frequency extraction step.

Next, foot strike and off time is detected to remove the effect of the floors. One can start off by computing the sum of wavelet coefficients over frequency within the extracted dominant frequency ranges, resulting in two time series. The higher range is for foot strike and the lower range is for foot off based on the floor types characterization supra. Then, peak-picking is conducted among the resultant wavelet coefficient time series to detect the time for foot strike and foot off. A reverse sliding window is applied starting from the peak to the valley to identify the time when the vibration starts to rise as the foot strike time. On the other hand, the peak of the lower frequency component determined as the foot-off time because it is when damped free vibration starts to attenuate the signal. Finally, each gait cycle is segmented based on the foot strike and foot-off time to compute the temporal gait parameters.

Finally, given the estimated foot strike time tᵢ^{s} and foot off time tᵢ^{o} for the i-th gait cycle. As described in FIG. 2, the gait parameters are estimated as follows:

Step Time=tᵢ₊₁^{s}−tᵢ^{s}

Stride Time=tᵢ₊₂^{s}−tᵢ^{s}

Stance Time=tᵢ^{o}−tᵢ^{s}

Swing Time=tᵢ₊₂^{s}−tᵢ^{o}

Single-Support Time=tᵢ₊₁^{s}−tᵢ₋₁^{o}

Double-Support Time1=tᵢ₋₁^{o}−tᵢ^{s}

Double-Support Time2=tᵢ^{o}−tᵢ₊₁^{s}

where t⁰ᵢ₋₁ is the previous gait cycle's foot off (i.e., opposite foot off) and t⁰ᵢ₊₁ is the next gait cycle's foot strike (i.e., opposite foot strike). For a given gait cycle, the single support time refers to the opposite swing phase. The first double support time is from the foot strike to the opposite foot off (the initial blue section at the opposite foot bar in FIG. 2), and the second double support time is from the opposite foot strike to the current foot off time.

### Floor-Agnostic Spatial Parameter Estimation

The spatial gait parameters that are estimated include step length, stride length, step width, step angle. These are estimated based on the footstep location during walking, which is important evidence to assess mobility, symmetry, and balance in gaits.

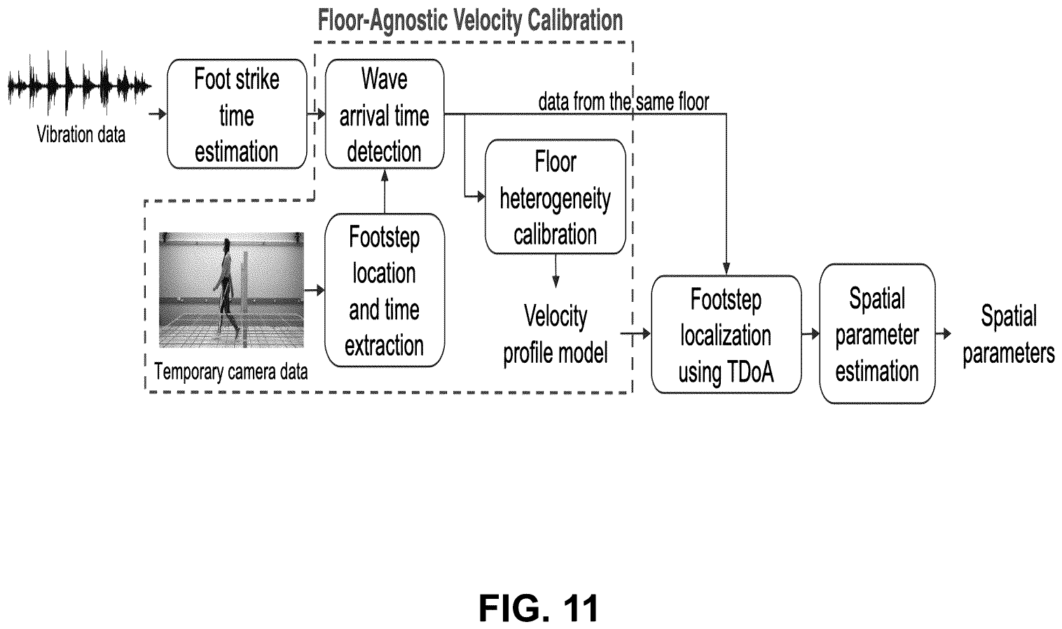

The approach for floor-agnostic temporal parameter estimation has four steps: 1) foot 414 strike time estimation, 2) floor-agnostic velocity calibration, 3) footstep localization, and 4) spatial parameter estimation. FIG. 11 shows the estimation process.

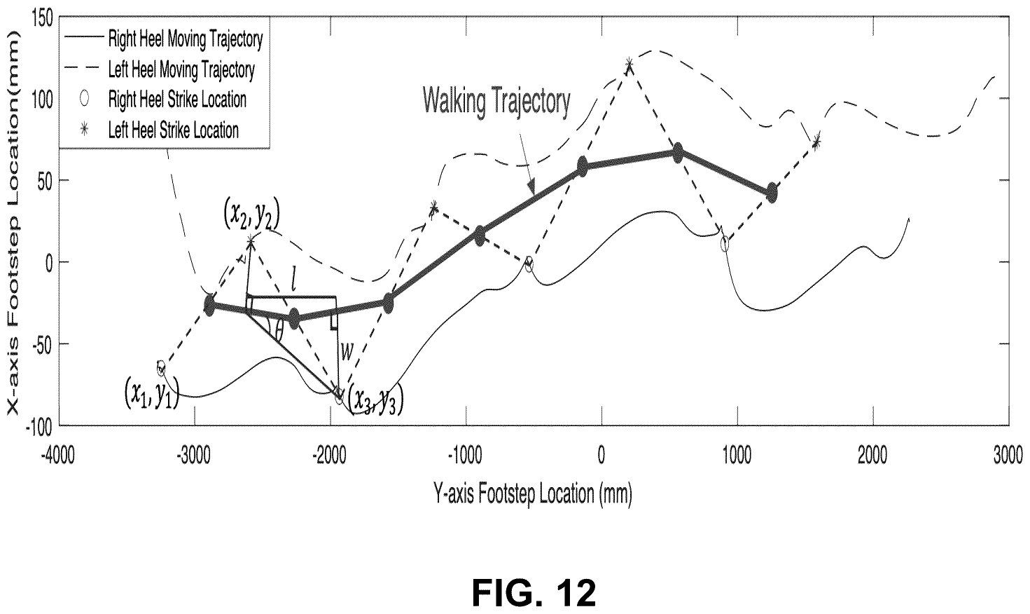

First, the time of foot strikes is estimated using the extracted the dominant frequencies discussed supra. This sets a foundation for wave arrival time detection. Then, the floor heterogeneity is calibrated caused by the variations in wave propagation velocity by setting up a temporary camera that records the step location and time for several walking trials, as introduced supra. Through a combined analysis of camera and vibration data, one then first estimates the wave arrival time by a peak-picking algorithm on signals between the foot-strike time and the time when the peak amplitude occurs in the high dominant frequency range. This is because the footstep force gradually increases after the initial contact with the floor, so the range of wave arrival time is always between the foot-strike time and the peak amplitude time. Then, one combines multiple sensors to finalize the wave arrival across sensors. Since the sensor closer to the footstep typically receives the wave first, one can then select the arrival time sequence based on the sequence of footstep-to-sensor distances. The wave propagation velocity profile is modeled based on the wave propagation distance and estimated wave arrival time at various footstep locations, as defined supra. Specifically, the velocity at each footstep location is computed by Equation 1. Assuming the wave propagation velocity is consistent at a given footstep location, the inventors model the velocity profile based on Equation 5 to reduce the influence of outliers and wave propagation directions. The output of the calibration process is a velocity profile model of the cross-sectional area of the floor structure. Next, the time difference of arrival (TDoA) is used across multiple sensors to estimate the footstep location. To achieve this, one first estimates the range of wave propagation velocity using the velocity profile model and the projected footstep location based on previous observations. Then, one computes TDoA over multiple sensors by subtracting the arrival time at the anchor sensor (the sensor with the largest signal amplitude). The location of the footstep is predicted through a grid search over the projected footstep range, where the location that leads to the lowest TDoA error is used. Finally, the gait parameters are according to FIG. 3. Since the walking trajectory of an individual may not be perfectly straight, the inventors estimated the walking trajectory for every three footsteps through a linear regression over the center points of the adjacent footstep locations. Given footstep (x1, y1), (x2, y2) and (x3, y3) described in FIG. 12, the first walking trajectory segment is estimated as:

$$y = {k}_{1} x + {b}_{1} ⇒ {k}_{1} = \frac{{y}_{1} - {y}_{3}}{{x}_{1} - {x}_{3}} , {b}_{1} = \frac{{y}_{1} + {y}_{2}}{2} - \frac{{x}_{1} + {x}_{2}}{2} \frac{{y}_{1} - {y}_{3}}{{x}_{1} - {x}_{3}} \qquad ( 6 )$$

where k₁ and b₁ describes gradient and interceptions for the 1-st walking trajectory segment. After repeating the calculation for all the walking trajectory segments, one can form a complete walking trajectory (marked as a thick green (now gray-scale) line in FIG. 12).

Then, each individual footstep is projected to the walking trajectory (see the projection for (x2, y2) and (x3, y3) in FIG. 12). Take the 3-rd footstep (x3, y3) as an example, the projection distance w is computed as the step width, and the distance between projected points l is computed as the step length. Based on trigonometry, the detailed calculation is summarized below:
Step Width:

$${w}_{i} = \frac{\vert {k}_{i - 1} {x}_{i} - {y}_{i} + {b}_{i - 1} \vert}{\sqrt{1 + {k}_{i - 1}^{2}}}$$

Step Length:

$${l}_{i} = ‖ ( \frac{{x}_{i - 1} + {x}_{i}}{2} , \frac{{y}_{i - 1} + {y}_{i}}{2} ) + {t}_{i} ( \frac{{x}_{i + 1} - {x}_{i - 1}}{2} , \\ \frac{{y}_{i + 1} - {y}_{i - 1}}{2} ) - [ ( \frac{{x}_{i} + {x}_{i + 1}}{2} , \frac{{y}_{i} + {y}_{i + 1}}{2} ) + {t}_{i + 1} ( \frac{{x}_{i + 2} - {x}_{i}}{2} , \frac{{y}_{i + 2} - {y}_{i}}{2} ) ] ‖$$

$$where {t}_{i} = \frac{( {x}_{i} - {x}_{i - 1} ) ( {x}_{i + 1} - {x}_{i - 1} ) + ( {y}_{i} - {y}_{i - 1} ) ( {y}_{i + 1} - {y}_{i - 1} )}{{( {x}_{i - 1} - {x}_{i + 1} )}^{2} + {( {y}_{i - 1} - {y}_{i + 1} )}^{2}}$$

θᵢ=tan⁻¹(ωᵢ/lᵢ) Step Angle:

sᵢ=lᵢ+lᵢ₊₁ Stride Length:

where the angle θi is approximated based on the step length and width. The stride length si is estimated by computing the sum of two adjacent step lengths.

### Gait Health Indicator Extraction

The gait health indicators we extract include cadence/walking speed, left-right symmetry, gait balance, and initial contact type, which reflect different aspects of the gait. In this section, the inventors describe the development of a quantitative scale on these indicators through floor vibration signals and discuss the physical insights behind each formulation.

### Cadence/Walking Speed Estimation

The cadence/step frequency is estimated by counting the number of footsteps per 10 seconds n₁₀ based on peak-picking on the sum of wavelet coefficients around the natural frequency range of the floor. For example, FIG. 9 (right) shows that there are n₁₀=12 peaks (i.e. footsteps) within the 10-second window, which means the step frequency is f=n/10=1.2 steps/second and the cadence is c=6n₁₀=72 steps/minute. The 10 measurement is only related to the temporal aspect of the gait. The walking speed, on the other hand, is related to both spatial and temporal information. In this approach, the walking speed vi is the step length divided by the step time, estimated as:

$${v}_{i} = \frac{{l}_{i}}{{t}_{i + 1}^{s} - {t}_{i}^{s}} \qquad ( 7 )$$

For example, if a person has a step length of 0.5 meters and a step time of 0.5 seconds, then the walking speed at that step is calculated as 1 m/s.

### Left-Right Symmetry Estimation

In this invention, the inventors focused on the left-right symmetry during the stance time. This is because the stance time is when the foot contacts the floor, which directly associates with the force transmission through the body, manifesting the left-right weight distribution. The inventors considered three aspects when assessing symmetry, including the 1) temporal, 2) spatial, and 3) kinetic measurements of the left and right foot. These correspond to the stance time, step length, and the signal energy normalized by the exponential of step-to-sensor distance.

With the above measurements, symmetry was described using the absolute symmetry index (SI) as introduced supra. This is because it does not require the classification of the left and right foot and focuses on the absolute difference between the two feet. The SI is defined as below:

$$SI = \frac{2 \vert {X}_{R} - {X}_{L} \vert}{{X}_{R} + {X}_{L}} \qquad ( 8 )$$

where X_{L} and X_{R} refer to the measurements of the left and right foot. In our approach, the stance time and the step length are used for temporal and spatial SI. The kinetic measurement (i.e., the ground reaction force) is represented by the normalized signal energy. This is because the inventors have found that the ground reaction force can be estimated through the signal energy compensated by the wave attenuation effect, which depends on the distance between the footstep and sensors.

### Gait Balance Quantification

As discussed supra, the approach describes gait balance based on the variability of walking to enable balance assessments in more realistic, non-clinical settings. Similar to the symmetry measurement, three aspects were considered when assessing balance, including 1) temporal, 2) spatial, and 3) kinetic measurements for balance, which corresponds to step time, step width, and the signal energy normalized by the exponential of the step-to-sensor distance. With the above measurements, gait balance is quantified by accumulating the difference between an individual footstep and the mean of all footsteps within the same trace. Specifically, the balance score (BS) is defined as follows:

$$BS = \sqrt{{\sum }_{i = 1}^{N} \frac{1}{N} {( \frac{{X}_{i} - \overline{X}}{\overline{X}} )}^{2}} \qquad ( 9 )$$

where Xi is the measurement of an individual footstep, X is the mean measurement of all footsteps within a trace, N is the number of footsteps in that trace. X corresponds to step time, step width, and normalized signal energy, respectively.

### Initial Contact Type Prediction

The initial contact type is predicted by a machine learning pipeline using frequency domain features discussed in supra. First, one takes the wavelet coefficients from the wavelet decomposition described supra to compute the coefficient sum over the frequency axis. Then, one divides the frequency axis into 10-Hz frequency bins and compute the mean of each bin as features to represent different types of contacts. Next, one trains a support vector machine model with a Gaussian kernel to capture the nonlinear dependencies among various frequency components and predict the initial contact type. To improve interpretability of the model predictions, one transforms the model confidence score using a softmax function to produce the probability of each class. To this end, the outcome of this data pipeline is the probability of each initial contact type, allowing further decision-making by human experts.

### Evaluation

To evaluation of the approach, the inventors conducted real-world experiments with 20 adults across concrete and wooden floors. First the experiment setup is discussed and then the results for spatiotemporal gait parameter estimation and gait health indicator extraction.

### Real-World Experiment

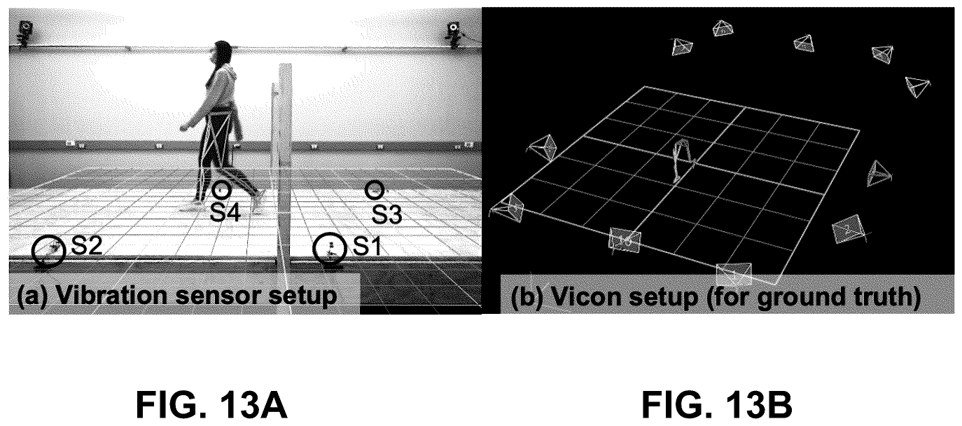

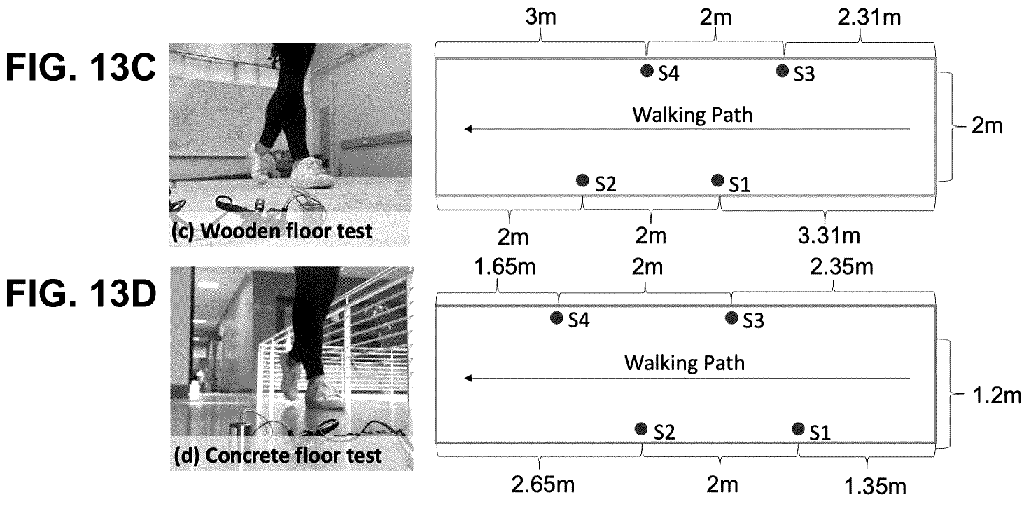

The experiment involves two sets of sensors: 1) eight geophone sensors mounted on the surface of the floor for vibration data collection, and 2) a Vicon motion capture system with 10 infrared cameras to record the ground truth of body movements during gait cycles. For each floor type, four sensors were installed at the side of the walking path, spaced 2 meters apart. FIGS. 13A-D show the experiment setup for both floors. The amplified analog signals are then converted into digital signals through NI-DAQ [51]. The sampling frequency is set to 25.6 kHz to maximize the temporal resolution of the vibration signals. The experiment involves 20 participants (aged from 18 to 40 years old) walking across one or two types of walkways using their normal gait, and each repeated for 40 trials back and forth. During each walking trial, 16 markers are attached to the subject's lower limbs, producing (x, y, z) coordinates of locomotion. The gait events are manually labeled, which include the “foot strike” and “foot off” time for each gait cycle. A total of 10,306 labeled gait cycles are collected and processed with ground truth on spatio-temporal gait parameters.

### Results

Overall, the approach has achieved an average of 92.5%, 87.6%, 92.3% accuracy in estimating temporal, and spatial gait parameters and gait health indicators, respectively. In this section, the inventors discuss the performance in these three categories and then show the gait profile from all testing participants to visualize the individual difference in gait patterns.

### Temporal Parameter Estimation Accuracy

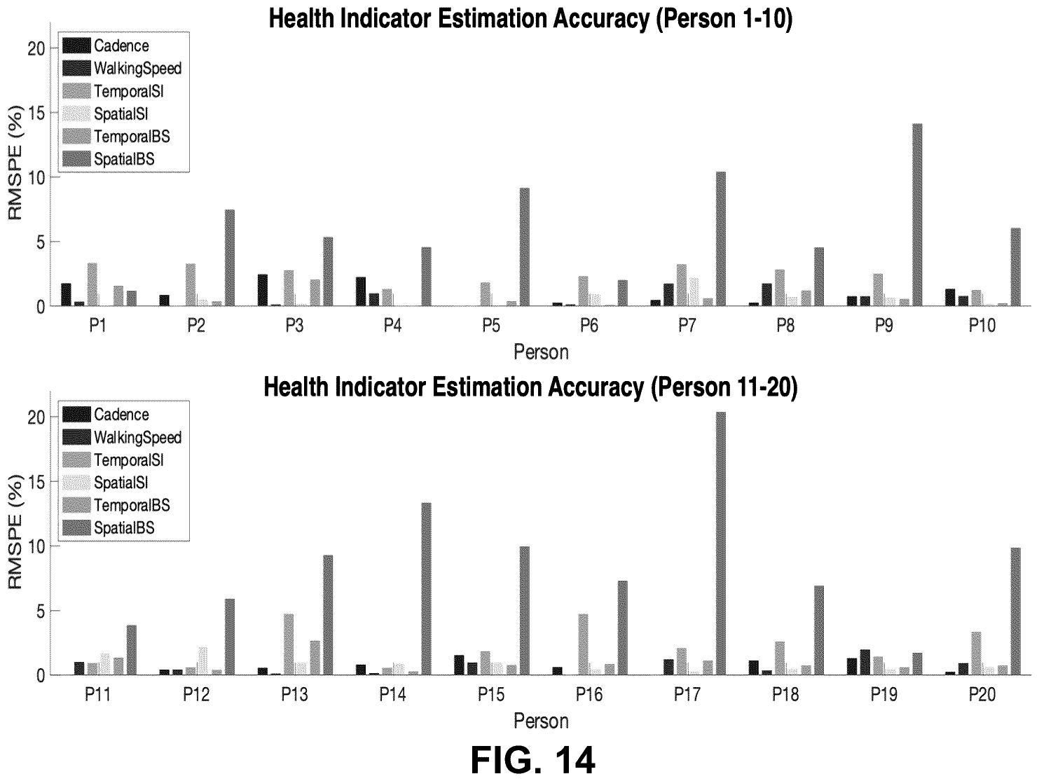

For temporal parameter estimation, the approach has an average of 0.08-second root-mean-square error (RMSE) among all subjects. Reader is referred to FIG. 14 in Appendix A in the priority document showing the detailed error rate for each parameter per person. Overall, the estimation errors are relatively consistent among all subjects. Persons 3, 12, and 15 have slightly larger errors compared to the rest of the subjects. This is because they have larger variations in temporal parameter values, leading to a less accurate estimation of dominant frequency ranges.

The error distribution among various types of parameters is also consistent across all subjects. In particular, stride time has the largest error due to the error accumulation in step time estimations. Double support time has the lowest error because it has the shortest duration among all (typically around 0.2 seconds). When one compares the RMS percentage error, the double support time has the largest error rate (around 20%) while the step time and stride time have the lowest error rate (around 7%).

### Spatial Parameter Estimation Accuracy

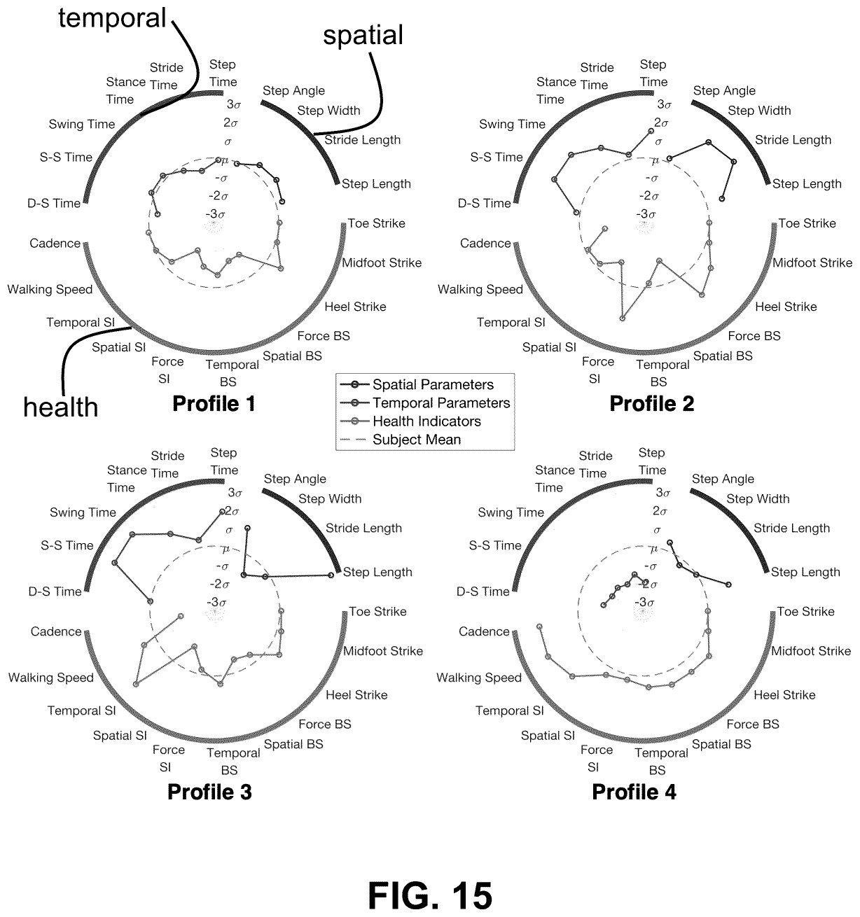

For spatial parameter estimation, the approach has an average of 0.38-meter length and 1.44-degree angle root-mean-square error (RMSE) among all subjects. Reader is referred to FIG. 15 in Appendix A in the priority document showing the detailed error rate for each parameter per person. Similar to the temporal parameter estimation, the errors for spatial parameters are also relatively consistent among all subjects. It was observed that persons 8, 9, and 19 have slightly larger errors than the rest of the subjects. This can result from the softer type of shoe they wear during the experiment while producing less impulsive signals during the initial contact, making it difficult to detect the exact time of wave arrival.

Interestingly, step location prediction has a significantly larger error than the step length estimation. This is because the localization error tends to bias towards the same direction due to the assumption of wave velocity across various directions. Therefore, the bias is mitigated by taking the Euclidean distance between the estimated locations of two adjacent footsteps. Among the spatial parameters, step width has the lowest RMSE due to its small value (typically around 0.15 meters). When comparing the RMS percentage error, the stride length has the lowest error rate (only around 5%) while the step width has a high (around 18%) error rate. While the step angle only has an RMSE of 1.44 degrees, the error rate of the step angle is high because the subjects were requested to walk in a straight line during the experiment, leading to small step angles in all recorded data.

### Gait Health Indicator Extraction Accuracy

For gait health indicator extraction, the approach has an average of 1 to 8% root-mean-square percentage error (RMSPE) among all subjects. As shown in FIG. 14. The majority of the errors are less than 5% except for the spatial BS. This is because the spatial BS is computed based on the estimated step width, which has a large error rate due to its relatively smaller value compared to the spatial resolution. Such error propagates into the BS estimation. In fact, the accuracy of gait health indicators estimation significantly relies on the accuracy of the temporal and spatial parameter estimation. In this evaluation, the inventors did not include the force SI, force BS, and contact type prediction because the ground truth (i.e., force measurements) is not available.

### Personalized Gait Profile

To visualize the gait parameter and gait health indicators among each individual, all the results above were summarized and personalized gait profiles were created for all human subjects. A personalized gait profile shows the deviation of each person's gait from the average gait among all people during the experiment, which provides a direct visualization for the person to understand the style of walking compared to the others. In addition, these profiles can also help with detecting gait abnormalities and tracking rehabilitation stages for patients.

FIG. 15 shows 4 typical profiles the inventors observed from 20 subjects.
Profile 1 “The Steady Walker”: This person's gait parameters are all within one standard deviation from the mean value. It means this person has a gait pattern that is close to the average of all walkers during the experiment. Also, the person has a low score for symmetry and balance, meaning that the person has good symmetry and stability.
Profile 2 “The Wide-Based Walker”: This person has a significantly larger step width than the rest of the subjects. As a result, the stride length and step time may also increase due to the wide base. On the other hand, the footstep forces are less symmetrical and balanced compared to the other subjects. This may be the root cause of the large step width because a wider base can typically help to maintain balance.
Profile 3 “The Large-Step Walker”: This person has a significantly larger step length and step time than the rest of the subjects. This means that the person takes large steps so that the during of each step also increases. As a result, the person still has a high walking speed while having a low cadence. Based on our record, this is the tallest person among all subjects, which explains this special gait profile.
Profile 4 “The Quick Walker”: This person has significantly smaller values in all temporal parameters while keeping the spatial parameters around the average. This means that the person takes medium steps but with quick left-right foot alternations. As a result, the person has a high cadence and high walking speed.

The inventors summarized the “subject mean” values of gait parameters from the study and compared them with the values from existing studies from a larger population, shown in the table below:

TABLE 1
Summary of “subject mean” gait parameters in our study
compared with existing studies.

| Gait Parameter | Mean (Ours) | Std (Ours) | Mean (Prev.¹) | Std (Prev.¹) |
|---|---|---|---|---|
| Walking Speed | 1.184 | 0.140 | 1.267 | 0.209 |
| (m/s) | | | | |
| Cadence | 104.1 | 8.566 | 114.0 | 9.300 |
| (step/min) | | | | |
| Step Time (s) | 0.581 | 0.046 | 0.541 | 0.043 |
| Stride Time (s) | 1.258 | 0.172 | 1.090 | 0.100 |
| Stance Time (s) | 0.747 | 0.066 | 0.632 | 0.045 |
| Swing Time (s) | 0.415 | 0.033 | 0.418 | 0.025 |
| Single-support | 0.415 | 0.033 | 0.415 | 0.025 |
| Time (s) | | | | |
| Double-support | 0.167 | 0.026 | 0.133 | 0.030 |
| Time (s) | | | | |
| Step Length (m) | 0.678 | 0.062 | 0.613 | 0.049 |
| Step Width (m) | 0.086 | 0.023 | 0.091 | 0.024 |
| Step Angle (°) | 4.123 | 1.287 | 4.290 | 1.800 |
| Stride Length | 1.415 | 0.214 | 1.398 | 0.150 |
| (m) | | | | |

As one can observe from Table 1, the mean and standard deviation from the data are consistent with several previous datasets with larger sample sizes. It is worth noting that the subjects in the data of this study have a slightly slower walking speed due to the larger step lengths and longer step time. Therefore, the “subject mean” used herein to generate gait profiles may bias towards a slower walking pattern.

Everyone has a unique gait profile observing from our data. Based on the record herein, the variations among the subjects' gait profiles are due to a mixture of complex reasons. For example, a person's height and weight are found to be correlated with the step length and time; a person's emotional status can affect the step frequency; also, the type of shoes a person is wearing can affect the entire gait profile. In addition, the inventors found that the left-right symmetry is affected by the leg length symmetry: there are 3 subjects that have asymmetrical left and right leg lengths (differ by around 1 inch), resulting in significantly higher SI and BS.

### Robustness Across Two Floor Types

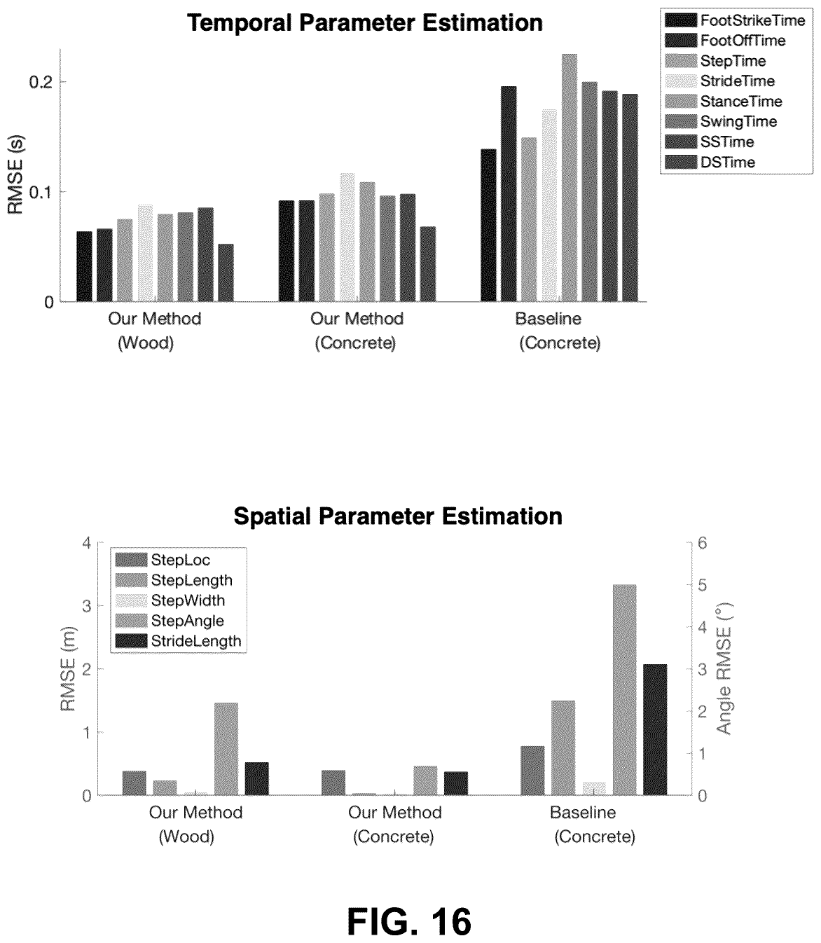

The approach has consistent results across two floor types based on the data from subjects who walked on both floors, which produces an average of 2.8× and 2.3× error reduction compared to the baseline. As shown in FIG. 16, the RMSE of the method on wood and concrete floors are significantly lower than that of the baseline method, despite that they follow similar trends among various parameters. The baseline method refers to the approach when there is no adaptation to floor types: 1) for temporal parameter estimation, the baseline does not consider the shift in dominant frequency ranges at foot strike and foot off, so it uses the same frequency range for the concrete floor as the wooden floor; 2) for spatial parameter estimation, the baseline does not estimate the velocity profile for the new floor and assumes the concrete floor has the same velocity profile as the wooden floor. The comparison shows that our approach is robust to various floor types.

What is claimed is:

1. A gait analysis method using footstep-induced floor vibrations, comprising:
(a) capturing floor vibration signals using two or more vibration sensors distributed and mounted on, within or under a floor, wherein the floor vibrations are footstep-induced floor vibrations caused by a person walking across the floor;
(b) predicting temporal gait parameters using a computer-implemented floor temporal estimation model with the captured floor vibration signals as input to the floor temporal estimation model;
(c) predicting spatial gait parameters using a computer-implemented spatial parameters estimation model with the captured floor vibration signals as input to the floor spatial parameters estimation model; and
(d) predicting gait health indicators using a computer-implemented gait health indicator extraction model with the predicted temporal gait parameters and the predicted spatial gait parameters as input to the implemented gait health indicator extraction model.

2. The method as set forth in claim 1, wherein the temporal gait parameters are step length, stride length, stance time, swing time, single stance time, double-support time, or a combination thereof.

3. The method as set forth in claim 1, wherein the spatial gait parameters are step length, stride length, step width, step angle, or a combination thereof.

4. The method as set forth in claim 1, wherein the gait health indicators are cadence, speed, symmetry, balance, initial contact, or a combination thereof.

5. The method as set forth in claim 1, wherein the gait health indicators are used to generate a personalized gait profile for the person to understand a gait health compared with an average gait from a group of people.
